(19) United States
(12) Patent Application Publication
(10) Pub. No.: US 2009/0035339 A1
(43) Pub. Date: Feb. 5, 2009

(54) Methods of Inactivating Viruses

(76) Inventors: Rudyard Istvan, Fort Lauderdale, FL (US); Michael Lynch, Hollywood, FL (US)

Correspondence Address: BRINKS HOFER GILSON & LIONE, P.O. BOX 10395, CHICAGO, IL, 60610, US

(21) Appl. No.: 11/869,512

(22) Filed: Oct. 9, 2007

Related U.S. Application Data
(60) Provisional application No. 60/851,028, filed on Oct. 10, 2006.

(51) Int. Cl. A61K 9/00; A61K 31/702; A01N 43/04

(52) U.S. Cl. 424/404; 514/57; 424/411

(57) ABSTRACT

A method of inactivating viruses is provided. The method comprises the step of topically applying an antimicrobial composition comprising: an organic acid; and an anionic surfactant mixture having a characteristic selected from the group consisting of: a linear alkyl chain having a chain length of from about C₄ to about C₁₂ and a total head group size of at least about 4 Angstroms; a branched alkyl chain having a chain length of from about C₄ to about C₁₂; an unsaturated alkyl chain having a chain length of from about C₄ to about C₁₂; and combinations thereof to an area in need of treatment.

### FIELD OF THE INVENTION

The present invention relates to methods of use of an antimicrobial composition to inactivate viruses. More specifically, the present invention relates to methods of use of an antimicrobial composition to inactivate non-enveloped viruses, such as caliciform viruses and more specifically norovirus.

### BACKGROUND

Human and mammalian health is certainly impacted by the spread of microbial entities at home, school, work and in the environment generally. Indeed, viruses and bacteria continue to cause a variety of sicknesses and ailments, prompting high absenteeism in schools and places of employment. In the wake of widespread food poisoning and the like, the public has become even further concerned with sanitization, both of person and property. Consequently, those of skill in the art have focused their research endeavors on the identification and deployment of suitable antimicrobial compositions, and specifically those that provide immediate and residual kill of microbes, with or without the use of water.

A comprehension of the vast benefits achieved via practice of the present invention requires an understanding of the various viruses against which the present methods are effective. Viruses may also be divided into two groups: enveloped and non-enveloped. Enveloped, or “lipophilic” viruses have an outer lipid-based membrane enveloping the capsid (comprised solely of capsomere proteins) that in turn protects the innermost viral genetic material. This enveloping membrane contains both viral and host cell proteins, and is acquired during budding from the host cell at the end of the viral replication process. Enveloped viruses include respiratory syncitial virus (RSV), and coronavirus, as well as influenza, measles and herpes simplex.

Non-enveloped, or “non-lipophilic” viruses do not have an enveloping membrane; their outer surface is the protein capsid. Such viruses include norovirus, rhinovirus, rotavirus, adenovirus, caliciform virus and hepatitis A. Non-enveloped viruses may be less susceptible to conventional antimicrobials than enveloped viruses. Typical antimicrobial agents such as alcohol that affect cell membranes may also effect the outer membrane of an enveloped virus, but may have little or no effect on the capsids.

Non-enveloped viruses are particularly difficult to adequately disinfect from environmental surfaces in general. Strong oxidizers like peracetic acids and bleaches inactivate all viruses with sufficient time and concentration, but they cannot be used on many surfaces without damaging them. Traditional disinfectants based on quaternary ammonium compounds (QACs) may have little or no effect on such viruses.

There are several contemporary compositions and methods for reducing and/or eliminating the formation of bacteria and/or viruses. For example, it is well known that the washing of hard surfaces, food (e.g. fruit or vegetables) and skin, especially the hands, with antimicrobial or non-medicated soap, is effective against viruses and bacteria. Actually, removal of the viruses and bacteria is due to the surfactancy of the soap and the mechanical action of the wash procedure, rather than the function of an antimicrobial agent. Thus, it is recommended that people wash frequently to reduce the spread of viruses and bacteria. However, many conventional products and methods of sanitization, including washing, fail to address the dilemma of sanitization “on the go”, that is to say, when a consumer is removed from the benefit of running water. Those skilled in the art have attempted to resolve this dilemma via the incorporation of antimicrobial agents into disinfecting lotions, cleansing wipes and the like. Such articles reduce the need for water during or following the application of the subject composition.

Other conventional antimicrobial cleansing products include deodorant soaps, hard surface cleaners, and surgical disinfectants. These traditional, rinse-off antimicrobial products have been formulated to provide bacteria removal during washing. A few such products, including antimicrobial soaps, have also been shown to provide a residual effectiveness against Gram-positive bacteria, but provide limited residual effectiveness against Gram-negative bacteria. By “residual effectiveness”, it is meant that the subject antimicrobial controls microbial growth on a substrate by either preventing growth of microbes or engaging in continuous kill of microbes for some period of time following the washing and/or rinsing process. To address the dilemma of limited residual efficacy against Gram-negative bacteria, those skilled in the art have sought to incorporate high levels of alcohol and/or harsh surfactants into contemporary antimicrobial products, which have been shown to cause dryness and irritation to skin tissues.

Furthermore, many of the conventional antimicrobial compositions that are capable of inactivating non-enveloped viruses can be harmful to skin, or harmful to surfaces where viruses such as norovirus are commonly found. By way of example, Hepacide® Quat II, a mixture of 4 QACs (quaternary ammonium compounds), claims EPA mandated levels of disinfection on hard, non-porous surfaces only for hepatitis B and hepatitis C with ten minutes of wet contact time. No effect on hepatitis A, which is the acute form, is claimed. Hepatitis B and C are enveloped viruses, while hepatitis A is a non-enveloped virus. Furthermore, Hepacide® Quat II and other QAC-based surface disinfectants are not safe for use on a user's skin, and normally carry labels that warn against contact with clothing or skin. Typically, if a QAC-based disinfectant does come into contact with skin, a user must remove it and wash the affected area with water for fifteen minutes, and then contact a poison control number for further instructions. Additionally, while these products are advertised as “skin-safe”, they can still damage skin if they are left in contact with skin for too long a period of time, or upon repeated application.

Other disinfectants that are effective against all viruses, such as RelyOn™, a multipurpose disinfectant cleaner distributed by DuPont™, must remain in contact with such viruses for a significant amount of time. RelyOn™ MDC is a peroxygen based powder that is designed to be prepared as a 1% solution in water. It is effective against a wide range of human pathogens, but a user is instructed to allow the solution to remain in contact with the viruses for ten minutes. It is not desirable to leave these solutions in contact with a surfaces that can be oxidized, such as wood, paint or fabric for such a lengthy period of time. Such solutions can deteriorate these materials if they remain in contact with them, much like bleach does. In fact, in less than 10 minutes, such compositions can damage brass and copper, and they can even damage stainless steel after longer periods of time.

Furthermore, antimicrobial compositions that exhibit rapid and residual kill of numerous bacteria and viruses have been disclosed in U.S. Patent Publication Nos. 2005/0271711, 2005/0260243, 2004/0001797 and 2003/0235550. Each of these disclosures are incorporated by reference herein. The compositions disclosed in these publications incorporate an organic acid or organic acid mixture, a specific short-chain anionic surfactant having at least one of a large, hydrophilic head group; an unsaturated structure; and/or a branched structure. They are adapted for direct application to human skin, without causing dryness or irritation. Moreover, they are designed for use with or without water, and provide immediate and residual effectiveness in either instance against a variety of viruses and bacteria, including rotavirus, rhinovirus, respiratory syncitial virus (RSV), coronavirus, Gram-positive and Gram negative bacteria. However, it has been surprisingly discovered that these compositions have a high log kill rate on caliciform viruses such as norovirus as well.

Norovirus is one of the most difficult viruses to disinfect. It is a member of the caliciform family that also affects other mammals including pets. Norovirus causes what is commonly known as “cruise-ship disease” and is the usual viral cause of acute gastroenteritis (AGE), accounting for 2/3 of all AGE cases, or 23 million cases annually, and 7% of all AGE deaths. The Center for Disease Control has noted the highly infective nature and persistence of norovirus, and that because of these traits, the transmission of norovirus is difficult to control through routine sanitary measures. The laboratory model for norovirus is FCV, or feline caliciform virus.

The need exists for a composition that can rapidly kill such viruses on all substrates, but also without damaging certain surfaces where they commonly reside. It would also be beneficial for such a composition to have long-lasting residual effects, so that the surfaces would remain free of active viruses long after the application of the composition to the surface.

### SUMMARY OF THE INVENTION

The present invention addresses and resolves all of the problems associated with the employment of conventional antimicrobial compositions and products to inactivate naked viruses, specifically norovirus. It has been surprisingly shown that the application of certain compositions to surfaces containing norovirus or surfaces that may come into contact with norovirus inactivates norovirus at an extremely high rate. Furthermore, these compositions do not have to remain in contact with the virus for a lengthy period of time to inactivate them, and they are not harmful to skin or porous surfaces.

Thus, in accordance with a first aspect of the present invention, a method of inactivating viruses is provided. The method comprises the step of topically applying an antimicrobial composition comprising: an organic acid; and an anionic surfactant mixture having a characteristic selected from the group consisting of: a linear alkyl chain having a chain length of from about C₄ to about C₁₂ and a total head group size of at least about 4 Angstroms; a branched alkyl chain having a chain length of from about C₄ to about C₁₂; an unsaturated alkyl chain having a chain length of from about C₄ to about C₁₂; and combinations thereof to an area in need of treatment.

In accordance with a second aspect of the present invention, a method of reducing the risk of viral infection and/or treating viral diseases in a mammal that may arise from said mammal's contact with a viral-infected surface is provided. The method comprises the steps of topically applying an antimicrobial composition comprising: an organic acid and an anionic surfactant mixture having a characteristic selected from the group consisting of: a linear alkyl chain having a chain length of from about C₄ to about C₁₂ and a total head group size of at least about 4 Angstroms; a branched alkyl chain having a chain length of from about C₄ to about C₁₂; an unsaturated alkyl chain having a chain length of from about C₄ to about C₁₂; combinations thereof to an area of said mammal that may or has come in contact with said surface; and optionally, removing said composition following said application.

In accordance with a third aspect of the present invention, a method of reducing inflammation in a mammal is provided. The method comprises the steps of topically applying an antimicrobial composition comprising: an organic acid and an anionic surfactant mixture having a characteristic selected from the group consisting of: a linear alkyl chain having a chain length of from about C₄ to about C₁₂ and a total head group size of at least about 4 Angstroms; a branched alkyl chain having a chain length of from about C₄ to about C₁₂; an unsaturated alkyl chain having a chain length of from about C₄ to about C₁₂; and combinations thereof to an inflamed area of said mammal in need of treatment.

In accordance with a fourth aspect of the present invention, a method of sanitizing mammalian skin is provided. The method comprises the step of topically applying an antimicrobial composition comprising: an organic acid and an anionic surfactant mixture having a characteristic selected from the group consisting of: a linear alkyl chain having a chain length of from about C₄ to about C₁₂ and a total head group size of at least about 4 Angstroms; a branched alkyl chain having a chain length of from about C₄ to about C₁₂; an unsaturated alkyl chain having a chain length of from about C₄ to about C₁₂; and combinations thereof to an area of mammalian skin.

In accordance with a fifth aspect of the present invention, a method of manufacturing an antimicrobial wipe is provided. The method comprises the steps of providing a substrate; and saturating said substrate with an antimicrobial composition comprising: an organic acid and an anionic surfactant mixture having a characteristic selected from the group consisting of: a linear alkyl chain having a chain length of from about C₄ to about C₁₂ and a total head group size of at least about 4 Angstroms; a branched alkyl chain having a chain length of from about C₄ to about C₁₂; an unsaturated alkyl chain having a chain length of from about C₄ to about C₁₂; and combinations thereof.

In accordance with a sixth aspect of the present invention, a method of manufacturing an antimicrobial drying towel is provided. The method comprises the steps of providing a substrate; saturating said substrate with an antimicrobial composition comprising: an organic acid and an anionic surfactant mixture having a characteristic selected from the group consisting of: a linear alkyl chain having a chain length of from about C₄ to about C₁₂ and a total head group size of at least about 4 Angstroms; a branched alkyl chain having a chain length of from about C₄ to about C₁₂; an unsaturated alkyl chain having a chain length of from about C₄ to about C₁₂; and combinations thereof; and removing all water from said substrate.

In accordance with a seventh aspect of the present invention, method of inactivating viruses is provided. The method comprises the step of: topically applying an antimicrobial composition comprising: from about 0.2% to about 70% of an organic acid and from about 0.1% to about 40% of an anionic surfactant mixture having a characteristic selected from the group consisting of: a linear alkyl chain having a chain length of from about C₄ to about C₁₂ and a total hydrophilic head group size of at least about 4 Angstroms; an unsaturated alkyl chain having a chain length of from about C₄ to about C₁₂; a branched alkyl chain having a chain length of from about C₄ to about C₁₂; and combinations thereof; wherein said composition is characterized by a pH of from about 2.0 to about 4.5; to an area in need of treatment.

### DETAILED DESCRIPTION OF THE INVENTION

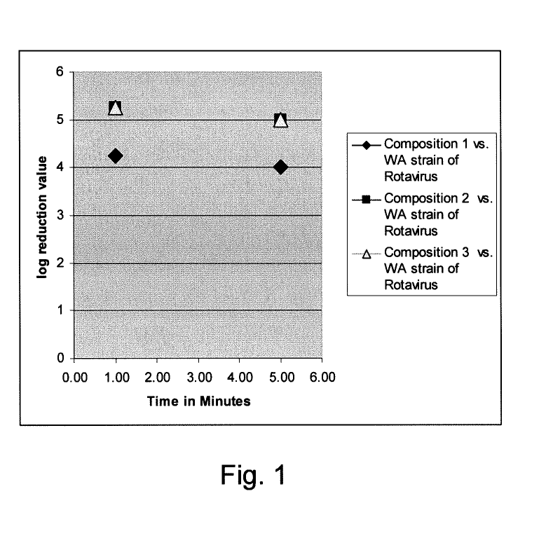

FIG. 1 is a graph showing the log reduction values of certain compositions versus a strain of Rotavirus.

### Antimicrobial Compositions

In accordance with the methods of the present invention, antimicrobial compositions, adapted for immediate and residual efficacy against a variety of bacteria and viruses, including caliciform viruses such as norovirus are provided. These compositions comprise an organic acid or organic acid mixture; an anionic surfactant having a chain length of from about C₄ to about C₁₂ and at least one of the following characteristics: an unsaturated structure, a branched structure; and/or a hydrophilic head group having a total head group size (defined, infra) of between about 4 to about 15 Angstroms. The compositions optionally further comprise a calcium ion scavenger and/or anti-foam agent. The compositions are characterized by a pH of between about 2.0 to about 4.5, depending on the specific constituents of the present antimicrobial compositions and the application for which their use is intended. Antimicrobial compositions and methods of making the compositions are taught and disclosed in U.S. Patent Publication Nos. 2005/0271711, 2005/0260243, 2004/0001797 and 2003/0235550, incorporated herein by reference.

### Organic Acid

The antimicrobial compositions for use with the methods of the present invention comprise an amount of an organic acid or organic acid mixture. Organic acids, for purposes of the present disclosure, are defined as proton-donating agents that remain at least partially undisassociated in a concentrated composition and remain so when the compositions are diluted during washing and rinsing. Without wishing to be bound by theory, the organic acids of the compositions serve to protonate the carboxylate functionalities on the phospholipid membrane of bacteria and reduce the tendency of the membrane to electronically repel anionic surfactants, thereby facilitating proper interaction between the present anionic surfactants and the membrane. With respect to viruses, the organic acids are believed to affect the lipid envelope and/or capsid in the same manner. Moreover, the organic acids disclosed herein facilitate the creation of a low pH buffer on the surface of a substrate, thereby prolonging the residual antimicrobial activity of the compositions and products in which they are incorporated.

Preferably, the organic acids are added directly to the compositions in acidic form or are formed by adding the conjugate base of the desired acid and an amount of a separate acid sufficient to form the undissociated acid from the base. The antimicrobial compositions for use with the method of the present invention comprise from about 0.2% to about 70%, preferably about 0.5% to about 40%, more preferably from about 1.0% to about 30%, and most preferably 0.1% to 10% based on the total weight of the antimicrobial composition, of an organic acid or organic acid mixture.

Suitable organic acids for use in the antimicrobial compositions include, but certainly are not limited to: pyroglutamic acid, adipic acid, gluconic acid, glyconolactone acid, glutamic acid, glycolic acid, glutaric acid, tartaric acid, ascorbic acid, benzoic acid, salicylic acid, citric acid, malic acid, succinic acid, lactic acid, carboxymethylcellulose and mixtures thereof. Other suitable organic acids for incorporation into the compositions are characterized by a pKa of greater than about 3.0. Without wishing to be bound by theory, the pKa selection limitation of the present organic acids serves the fundamental goal of ensuring that at least 50% of the organic acids incorporated into these compositions remain undissociated at the desired pH of from about 2.0 to about 4.5 (discussed, infra).

### Optional Calcium Ion Scavenger

The antimicrobial compositions for use with the methods of the present invention can further comprise a calcium ion scavenger. Without wishing to be bound by theory, the calcium ion scavengers facilitate the disruption of the cell membrane of bacteria by the anionic surfactants via capture of the calcium ions of the phospholipid cell membrane. With respect to viruses, the calcium ion scavengers are believed to affect the lipid envelope and/or capsid in the same manner. Without wishing to be bound by theory, said calcium ions are believed to exist within and around the cell membrane, thereby often preventing the penetration of conventional surfactants. Suitable calcium ion scavengers of the present invention, include, but are not limited to: citric acid, malic acid, succinic acid, polyacrylic acid, copolymers of acrylic acid and maleic acid, oxydisuccinic acid, nitrilotriacetic acid, iminodisuccinic acid, tartrate disuccinic acid, tartrate monosuccinic acid, ethylenediaminetetraacetic acid, pyrophosphoric acid and mixtures thereof. The antimicrobial compositions for use with the method of the present invention comprise preferably from about 0.1%-3.0%, based on the total weight of the antimicrobial composition, of a calcium ion scavenger or a calcium ion scavenger mixture.

In another aspect of the compositions for use in the methods of the present invention, the calcium ion scavengers are characterized by a pKa of lower than about 3.0. Moreover, in another aspect of the compositions, suitable calcium ion scavengers are characterized by a calcium ion binding constant (log P) of greater than about 3.0 at a pH of about 3.

### Anionic Surfactant

The anionic surfactants in the compositions for use with the methods of the present invention have a chain length of from about C₄ to about C₁₂ and at least one characteristic selected from: a large hydrophilic head group; an unsaturated structure, and/or a branched structure; these anionic surfactants provide enhanced performance benefits, while minimizing dryness and/or irritation to mammalian skin tissue. These short chain anionic surfactants exhibit phase stability in formulation, compatibility with other antimicrobial agents and residual efficacy of the antimicrobial compositions in which they are incorporated. Without wishing to be bound by theory, it is believed that the interaction of short chain anionic surfactants with the phospholipid cell membrane of bacteria, facilitated by the protonation of carboxylate functionalities at the surface of the membrane, disrupts the membrane and denatures cellular proteins, thereby providing rapid microbiocidal activity. With respect to viruses, the short chain anionic surfactants are believed to affect the lipid envelope and/or capsid in the same manner.

The antimicrobial compositions for use with the methods of the present invention comprise from about 0.1% to about 40% preferably from about 0.2% to about 30%, more preferably from about 0.3% to about 20%, and most preferably from about 0.1%-3.0% of an anionic surfactant mixture. In another aspect of the compositions, the short-chain anionic surfactants disclosed herein are incorporated into the antimicrobial compositions at a level of greater than about 25%. The anionic surfactants useful for incorporation into these antimicrobial compositions comprise a relatively short carbon chain, preferably between about C₄ to about C₁₂, more preferably between about C₆ to about C₁₁, most preferably between about C₆ to about C₁₀. It should be noted, however, that, due to the fact that some surfactants suitable for incorporation into the present antimicrobial compositions are commercially available in mixed chain lengths, the average chain length of the resultant anionic surfactant mixture may differ from the above-described ranges.

To reiterate, those of skill in the art have generally avoided the incorporation of so-called “short chain” anionic surfactants into antimicrobial compositions. This trend is believed to be due in part to the conventional wisdom in the art that short chain anionic surfactants are characterized by decreased interfacial activity and decreased interaction with the phospholipid membrane of bacteria and the lipid envelope of enveloped viruses, and thus, provide poor microbiocidal activity. Accordingly, those of skill in the art have generally relied upon the employment of anionic surfactants with chain lengths of from C₁₂ to C₁₆ in antimicrobial compositions. The chain lengths of such surfactants are comparable to those of the acyl components in the phospholipid membrane of bacteria and the lipid envelope of enveloped viruses, and thus, are thought to provide optimum microbiocidal activity. Moreover, longer chain surfactants have conventionally been thought to be less capable of skin penetration, and thus, less likely to cause dryness and irritation to skin. Nevertheless, conventional, longer chain anionic surfactants often exhibit poor phase stability in an acidic product matrix, incompatibility with cationic antimicrobial agents and decreased residual antimicrobial activity. Conversely, the shorter chain anionic surfactants used in the compositions for use with the methods of the present invention exhibit surprisingly high immediate microbiocidal activity, phase stability in broad concentration ranges of acidic aqueous matrices and compatibility with cationic antimicrobial agents. Importantly, the anionic surfactants used in the compositions for use with the methods of the present invention prevent dryness or irritation to skin and demonstrate strong residual microcidial activity on a target substrate when the substrate is later inoculated with bacteria or virus.

In another aspect of the antimicrobial compounds for use with the methods of the present invention, the short chain anionic surfactants disclosed herein possess an unsaturated structure and/or a branched, hydrophobic group with a total carbon content ranging from about C₄ to about C₁₂, preferably from about C₆ to about C₁₁ and more preferably from about C₆ to C₁₀. In yet another aspect of the compounds, the short-chain anionic surfactants disclosed herein comprise a hydrophilic head group with a total head group size of less than about 15 Angstroms, preferably less than about 10 Angstroms, more preferably between about 4 to about 7 Angstroms. By “total head group size,” it is meant the accumulated size of every substituent on the hydrophilic head group of the present anionic surfactants. That is to say, the present anionic surfactants may comprise more than one substituent on their subject hydrophilic head groups, for a combined, total hydrophilic head group size falling within the above-listed ranges. Without wishing to be bound by theory, it is believed that the unsaturated structure and/or branched structure and/or large hydrophilic head group of the present anionic surfactants increases their water solubility, increases their compatibility with cationic agents, increases steric hindrance to their disruption of the stratum conium layer of skin and maintains their substantivity to the phospholipid membrane of bacteria and the lipid envelope and/or capsid of viruses.

The “hydrophilic head group” is defined as the hydrophilic portion (which may contain both non hydrocarbon and hydrocarbon units) of the anionic surfactant, measured from the first polar atom to the end of the hydrophilic segment that links to the hydrophobic body. For example, the hydrophilic head group of alkyl glyceryl sulfonate R—O—CH₂CH(OH)CH₂—SO₃Na is —O—CH₂CH(OH)CH₂—SO₃Na. The hydrophilic head group size is estimated from the Van der Waals radius of the atoms and the configuration of the surfactant molecule. Suitable hydrophilic head groups of the present invention with a size of less than about 10 Angstroms include, but are not limited to: glyceryl ether sulfonates and, for compositions having a pH of greater than 3.5, isethionates, sulfosuccinates, amidosulfonates and ethoxylated sulfonates.

In yet another aspect of the compounds, the head group of the anionic surfactant is characterized by substitution of one or more substituents. By “substituents” it is meant any hydrophilic segment that is bonded to the head group, defined hereinbefore, of the present anionic surfactants. Without wishing to be bound by theory, it is believed that such increased substitution on the head group of the present anionic surfactants further increases the size and hydrophilicity of the head group. Suitable hydrophilic head groups of the compositions with multiple substituents include, but are not limited to, alpha sulfo fatty acid, and if the pH of the present antimicrobial compositions is greater than 3.5, monoester of sulfosuccinic acid. To reiterate, the head group size of the present anionic surfactants is defined on the basis of Angstroms, as discussed supra. Thus, although the hydrophilic head group of the present anionic surfactants may comprise more than one substituent, the total hydrophilic head group size should not exceed the preferred size ranges, set forth hereinbefore, in Angstroms.

Accordingly, suitable anionic surfactants of the compositions, meeting all of the criteria discussed hereinbefore include, but certainly are not limited to: linear or branched alkyl glyceryl sulfonate, alkyl alpha sulfo fatty acid, alpha olefin sulfonate, branched alkyl sulfonate, branched alkyl benzene sulfonate, branched alkyl phosphonate and if the pH of the antimicrobial composition is greater than about 3.5, secondary alkyl sulfate, alkyl isethionate, monoester of alkyl sulfosuccinic acid, alkyl aminosulfonate, alkyl ethoxylated sulfonate, and combinations thereof. The aforementioned list is only intended to serve as a guide to the formulator of the antimicrobial compositions. Additional anionic surfactants having a chain length of from about C₄ to about C₁₂ and comprising at least one of the following characteristics are suitable for use herein: an unsaturated structure; a branched structure and/or a hydrophilic head group size as described hereinbefore. Selection of the appropriate anionic surfactant for use in the antimicrobial compositions will depend upon the needs and/or abilities of the formulator. Other surfactants, many commercially available, are incorporated into the antimicrobial compositions. For example, in the experiments described infra, the anionic surfactants were C8AGS [CAS 51946-14-6] and C8-10 MES (methyl ester sulfonate). Said surfactants, although depending on the precise form of the desired antimicrobial composition, include, but certainly are not limited to: paraffin sulfonate, hydrolyzed methyl aster sulfonate, alkyl sulfosuccinate, alkyl glyceryl sulfonate, alpha olefin sulfonate, alkyl isethionate, secondary alkyl sulfate, branched alkyl benzene sulfonate, alkyl sulfate and combinations thereof.

It should be noted and underscored that selection of the appropriate anionic surfactant for use in the context of the antimicrobial compositions will depend upon several factors, including, but certainly not limited to: the nature of the substrate for which use of the antimicrobial compositions disclosed herein is desired and the needs and/or abilities of the formulator and/or practitioner of the present compositions. For instances in which the mildness of the present antimicrobial compositions on skin is not an issue, short chain anionic surfactants having a hydrophilic head group size of less than about 4 Angstroms and/or a linear structure may be suitable for use in the context of the present invention. Indeed, for instances in which mildness of the present compositions on skin is not a fundamental concern, suitable anionic surfactants for use in the context of the present invention include, but certainly are not limited to: sulfonates and sulfates having a linear chain with a chain length of from about C₄ to about C₁₂, preferably having a chain length of from about C₆ to about C₁₂, more preferably having a chain length of from about C₈ to about C₁₂.

### Anti-Foam Agent

The antimicrobial compositions for use with the methods of the present invention may also comprise an anti-foam or suds suppression agent. Incorporation of said agents is particularly desired for applications in which the antimicrobial compositions comprise high sudsing, short chain anionic surfactants such as alkyl glyceryl sulfonate and/or a level of anionic surfactant of greater than about 1 weight percent. Incorporation of an anti-foam agent or suds suppression system is further advantageous in compositions for which low foaming is desired, particularly when such foaming has the affect of decreasing the conveyance of antimicrobial dosage. The antimicrobial compositions comprise an anti-foam or suds suppression agent, present at a level of from about 0.0001% to about 15%, preferably from about 0.001% to about 10%, most preferably from about 0.005% to about 5.0% by weight of the antimicrobial composition. The anti-foam agent is present in an amount of at least 1 ppm by weight of the total composition. Without wishing to be bound by theory, it is believed that incorporation of an anti-foam agent or suds suppression system serves the fundamental goal of controlling the suds profile of the present compositions during production and ensuring the delivery of an optimum dosage of the present antimicrobials during employment. Indeed, suitable suds suppressing systems for use herein may comprise essentially any known anti-foam compound that exhibits stability at a pH of about 2.0 to about 4.5, including, but not limited to, those selected from the group consisting of silicone anti-foam compounds, silicone emulsions, 2-alkyl and alkanol anti-foam compounds, mineral oil emulsions, hydrocarbon oil emulsions, polyalkylene emulsions and combinations thereof.

Silicone suds suppressor technologies and other anti-foam agents useful herein are extensively documented in “Defoaming, Theory and Industrial Applications”, Ed., P. R. Garrett, Marcel Dekker, N.Y., 1973, ISBN 0-8247-8770-6, incorporated herein by reference. See especially the chapter “Surfactant Antifoams” (Blease et al). See also U.S. Pat. Nos. 3,933,672 and 4,136,045, both incorporated herein by reference. Highly preferred silicone suds suppressors are the compounded types known for use in antimicrobial compositions, including, for example, polydimethylsiloxanes having trimethylsilyl or alternate endblocking units. Such compounds may be compounded with silica and/or with surface-active nonsilicon components, as illustrated by a suds suppressor comprising 12% silicone/silica, 18% stearyl alcohol and 70% starch. A suitable, commercial source of the silicone active compounds is Dow Corning Corp.

### Optional Nonionic Agent

The antimicrobial compositions disclosed herein for use with the methods of the present invention may further comprise a nonionic agent. Suitable nonionic agents for use in the compositions are selected from the group consisting of: alkyl polyols, alkyl alcohols, phenols, chloro phenols, polyphenols and mixtures thereof. Without wishing to be bound by theory, it is believed that the optional nonionic agent of the composition serves many roles, including, but certainly not limited to, increasing the antibacterial efficacy, in both immediate and residual kill, of the organic acid and short chain anionic surfactant system of the present invention. Some alkyl polyols, such as 1-(2-ethylhexyl)glycerol ether, have conventionally been thought to inhibit bacteria, and thus, have traditionally been employed as preservatives in commercial cosmetic products. Use of alkyl polyols and alkyl alcohols in these compositions has the affect of increasing their immediate and residual activity. When present, the nonionic agents are incorporated into the antimicrobial compositions in an amount of from about 0.1% to about 10%, preferably from about 0.1% to about 5.0% more preferably from about 0.1% to about 3.0%, by weight of the total antimicrobial composition. When the antimicrobial compositions comprise a nonionic agent, said agent comprises a carbon chain length of from about C₃ to about C₁₂. Suitable nonionic agents for incorporation into the antimicrobial compositions include, but certainly are not limited to: 1-(2-ethylhexyl) glycerol ether, octyl glycerol ether, 2-(2-ethylhexylxoxy) propanol (EHOP), octyloxy propanol, 1-(2-ethylhexyloxy) ethanol, octyloxy ethanol, 1,2 hexylenediol, 1,2-cyclohexanedimethanol, isopropyl glycerol ether, 4-chloro-3-xylenol and combinations thereof. In another aspect of the compositions, the nonionic agent is branched, unsaturated or linear. In yet another aspect of the compositions, the nonionic agent is substituted with compounds selected from the group consisting of: alcohols, polyols, phenols, chloro phenols, polyphenols and combinations thereof.

### Optional Adjunct Ingredients

In another aspect of the compositions for use with the methods of the present invention, the compositions may comprise one or more adjunct ingredients. Said ingredients maybe employed to increase the mildness of the desired composition, increase immediate and/or residual efficacy of the subject compositions, improve the wetting characteristics of the subject compositions upon application to a target substrate, operate as solvents for diluted compositions, and/or serve to modify the aesthetic characteristics of the composition. The compositions may comprise from about 0% to about 70%, preferably from about 0% to about 62%, more preferably from about 0% to about 10%, of an alcohol solvent. Suitable alcohol solvents include, but are not limited to, ethanol, propanol, butanol, probpylene glycol, diethylene glycol, dipropylene glycol and mixtures thereof.

In another aspect of the compositions for use with the methods of the present invention, the compositions may comprise from about 0% to about 10% preferably from about 0% to about 5%, more preferably from about 0% to about 1%, of a cationic antimicrobial agent. Depending on the region in which the formulator chooses to practice the present methods, the inclusion of one or more cationic surfactants may be necessary for the procurement of regulatory approval. Suitable cationic antimicrobial agents for use in the compositions include, but certainly are not limited to, benzalkonium chloride, benzethonium chloride, triclocarban, tricolsan, chlorhexidine and mixtures thereof.

The compositions disclosed herein comprise from about 0% to about 5%, preferably from about 0% to about 2%, of a heavy metal salt selected from the group consisting of: silver, zinc, copper and mixtures thereof. Incorporation of said heavy metal salt serves to increase the antimicrobial activity and the viscosity of these antimicrobial compositions. Moreover, the other ingredients of the present compositions have exhibited compatibility with the heavy metal salts disclosed herein. The compositions disclosed herein comprise from about 0% to about 20% preferably from about 0% to about 5%, of a skin emollient or moisturizer. Such ingredients serve the fundamental purpose of increasing the mildness (discussed infra) of the present antimicrobial compositions and are particularly desired when incorporating the present antimicrobial compositions into a skin care product (discussed infra).

### pH of Antimicrobial Compositions

It is fundamental to achieving the benefits of the methods of the present invention that the undissociated acid from the organic acids disclosed in the compositions remain on the skin in the protonated form. Thus, the pH of the antimicrobial compositions must be adjusted to a sufficiently low level in order to either form or deposit substantially undissociated acids onto the substrate for which treatment is desired. By “substantially undissociated,” it is meant that, upon application of the present compositions onto a target substrate, such as mammalian skin, about 30%, preferably 50%, more preferably 70%, of the organic acids incorporated in said compositions remain undissociated following the lapse of about 30 minutes from application. The pH of the present compositions should be adjusted and preferably buffered to achieve the desired range. The antimicrobial compositions disclosed herein are characterized by a pH of from about 2.0 to about 4.5, preferably from about 2.5 to about 4.0. Indeed, the pH of the antimicrobial compositions will depend upon the precise ingredients incorporated into the subject compositions. Nevertheless, the pH of the compositions is generally, and preferably, above about 2.0, as compositions characterized by a pH below 2.0 are typically required to be identified as toxic or hazardous materials.

### Mildness of Antimicrobial Compositions

Topically applied products, including rinse-off cleansers and leave-on sanitizers, have conventionally possessed the tendency to irritate or dry mammalian skin. The compositions for use with the methods of the present invention, however, provide immediate and residual kill of bacteria and viruses, while possessing the fundamental characteristic of mildness. By “mildness” it is meant the degree to which a composition prevents dryness or irritation to skin. Factors that influence the mildness of a topically applied antimicrobial product include, but are not limited to, duration of exposure to the product, the frequency of use of the product and the degree to which the skin is occluded following exposure to the product.

Irritation is observed by several methods, including but not limited to, visual and instrumental assessment of the erythema for redness and of the skin for edema following application of an antimicrobial product. Irritation may be measured by determining the transepidermal water loss (TEWL of skin before and after exposure to an antimicrobial product, using, for example, a TEWL meter. Indeed, products that cause irritation may eventually compromise the natural barrier function of mammalian skin—resulting in increased water loss through the epidermis. Dryness is observed by several methods including, but not limited to, visual and instrumental assessment of the level and severity of dry skin flakes following exposure to an antimicrobial product. Dryness may be measured by instruments that examine the water content of the skin. One such instrument, a corneometer, measures the water content of skin via capacitance.

The compounds for use in the methods of the present invention, despite their enormous cleaning and antimicrobial characteristics, are adapted to ensure increased mildness to mammalian skin upon application, particularly when compared to conventional cleansers such as bar or liquid soap and leave-on sanitizers. Indeed, the efficacy and mildness of these compositions has been examined and illustrated under a variety of use conditions and methods. Namely, during a 10-day clinical forearm study, subjects applying these compositions experienced significantly less skin irritation and dryness than subjects engaging in the same number of washes per day with soap and water and subjects applying conventional alcohol-based hand sanitizers. The results of the aforementioned study were measured using both visual and instrumental methods. The 10-day clinical forearm study is intended to mirror the hand washing and/or sanitizing use frequency typically recommended for proper hygiene. In another study, the leave-on application of these compositions was applied 4 times daily, in addition to normal hand washing, and resulted in no measurable skin irritation or dryness.

### Products Incorporating Antimicrobial Compositions

### Personal Care Products

The methods of the present invention can be performed in a variety of ways. For example, personal care products containing the antimicrobial compositions are disclosed. These personal care products can be used to disinfect areas that have come into contact with, or may come into contact with, non-enveloped viruses such as norovirus. Suitable personal care products include, but are not limited to: hand soaps, hand sanitizers, body washes, mouth washes, toothpastes, shower gels, shampoos, body lotions, deodorants, nasal sprays, foot care, vaginal care and/or wash, pet care and combinations thereof. The personal care products disclosed herein take the form of a wipe product, particularly suitable for wiping or drying the face or hands. In such instance, the antimicrobial compositions are preferably embedded or impregnated into said wipe product. In yet still another aspect of the present invention, the personal care product disclosed herein takes the form of a tissue or towel, also suitable for wiping or drying the face or hands. Such a dry towel can be used to disinfect wet surfaces that have come into contact with, or may come into contact with non-enveloped viruses, such as norovirus. The method of the present invention could also be performed with a personal care product in the form of a first aid antiseptic for irritated, injured, or acne-affected skin and/or for pre or post surgical use.

### Household Care Products

The methods of the present invention can also be practiced utilizing the disclosed compositions incorporated into one or more household care products. Indeed, suitable household care products for use with the methods of the present invention include, but are not limited to: hard surface cleaners, deodorizers, fabric care compositions, fabric cleaning compositions, manual dish detergents, automatic dish detergents, floor care compositions, kitchen cleaners or disinfectants, bathroom cleaners or disinfectants and combinations thereof. The household care product can take the form of a wipe or towel, suitable for household cleaning and/or care. The household care products disclosed herein can also comprise certain adjunct ingredients. Said adjuncts include, but certainly are not limited to: detersive enzymes, builders, bleaching agents, bleach activators, transitional metal bleach catalysts, oxygen transfer agents and precursors, soil release agents, clay soil removal and/or anti-redeposition agents, polymeric dispersing agents, brightener, polymeric dye transfer inhibiting agents, chelating agents, anti-foam agents, alkoxylated polycarboxylates, fabric softeners, perfumes, carriers, hydrotropes, processing aids, dyes or pigments, solvents for liquid formulations, solid fillers, detersive surfactants and combinations thereof.

### Commercial Disinfecting Products

The methods of the present invention can also be practiced utilizing the disclosed compositions incorporated into one or more commercial disinfecting products. Such products are useful in disinfecting restaurants, nursing homes, cruise ships, public restrooms, offices, and the like. Indeed, suitable commercial disinfecting products for use with the methods of the present invention include, but are not limited to: hard surface cleaners, deodorizers, fabric care compositions, fabric cleaning compositions, manual dish detergents, automatic dish detergents, floor care compositions, kitchen cleaners or disinfectants, bathroom cleaners or disinfectants and combinations thereof. The commercial disinfecting product can take the form of a wipe or towel, suitable for commercial cleaning and/or disinfecting. The commercial disinfecting products disclosed herein can also comprise certain adjunct ingredients. Said adjuncts include, but certainly are not limited to: detersive enzymes, builders, bleaching agents, bleach activators, transitional metal bleach catalysts, oxygen transfer agents and precursors, soil release agents, clay soil removal and/or anti-redeposition agents, polymeric dispersing agents, brightener, polymeric dye transfer inhibiting agents, chelating agents, anti-foam agents, alkoxylated polycarboxylates, fabric softeners, perfumes, carriers, hydrotropes, processing aids, dyes or pigments, solvents for liquid formulations, solid fillers, detersive surfactants and combinations thereof.

### Skin Care Products

The methods of the present invention can also be practiced utilizing the disclosed compositions incorporated into one or more skin care products. The skin care products can incorporate a dermatologically acceptable carrier to facilitate safe transfer of the antimicrobial composition to the desired area of the skin. The skin care product of can also comprise certain adjunct ingredients. Said adjuncts include, but certainly are not limited to: antimicrobial actives and antifungal actives such as parachlorometazylenol (PCMX) or potassium sorbate, surfactants, desquamation actives, anti-acne actives, anti-wrinkle actives, anti-atrophy actives, anti-oxidants, radical scavengers, chelators, flavonoids, anti-inflammatory agents, anti-cellulite agents, topical anesthetics, tanning actives, sunscreen actives, conditioning agents, thickening agents, detackifying agents, odor control agents, skin sensates, antiperspirants and mixtures thereof. Indeed, a complete description and examples of each of the aforementioned adjunct ingredients is set forth in U.S. Pat. No. 6,294,186, assigned to The Procter and Gamble Company, Cincinnati, Ohio and incorporated herein by reference.

### Articles of Manufacture & Kits

The methods of the present invention can also be practiced utilizing the disclosed compositions incorporated into articles of manufacture containing the antimicrobial composition and/or one or more of the aforementioned products. These articles of manufacture are intended for personal care, skin care and household care applications. These articles of manufacture encompass one or more products as described hereinbefore that may be packaged in a container or dispenser with a set of instructions for the consumer. The articles of manufacture typically comprise (a) container or dispenser, (b) product and (c) set of instructions to apply said product to an appropriate substrate to achieve immediate and residual antimicrobial activity. Containers and/or dispensers suitable for the article of manufacture of the present invention include, but are not limited to: PET bottles and tubs, flow-wrap pouches, foaming dispensers, spray dispensers and combinations thereof. To reiterate, the articles of manufacture for use in practicing the method of the present invention further comprise a set of instructions in association with the container. By “in association with,” it is meant that the instructions are either directly printed on the container or dispenser itself or presented in a different fashion including, but not limited to: a brochure, print advertisement, electronic advertisement and/or verbal communication, so as to communicate the set of the instructions to a consumer of the article of manufacture.

The set of instructions typically comprise the instructions relating to the use of the product to apply the antimicrobial composition onto a suitable substrate for which treatment is sought. The set of instructions may further comprise the instruction to allow the antimicrobial composition to remain on the treated substrate, without rinsing or otherwise removing the antimicrobial composition from the treated substrate. Nevertheless, the precise instructions included with the articles of manufacture will depend on the precise ingredients of the subject antimicrobial composition and the product for which the inclusion of instructions is desired and the substrate onto which application of the product is intended.

### Methods of Use

The methods of the present invention are suitable for a variety of uses. Indeed, suitable uses include, but certainly are not limited to, the inactivation of viruses; the inactivation of non-enveloped viruses, the provision of residual anti-viral efficacy; the inactivation of norovirus; the prevention of disease caused by norovirus; the sanitization of hard surfaces; the improvement of the overall health of a mammal; the reduction of absenteeism; and combinations thereof.

Indeed, in one aspect of the present invention, a method of inactivating viruses is provided. The method comprises the steps of topically applying a composition comprising an organic acid and an anionic surfactant mixture having a characteristic selected from the group consisting of:
a. a linear alkyl chain having a chain length of from about C₄ to about C₁₂ and a total head group size of at least about 4 Angstroms;
b. a branched alkyl chain having a chain length of from about C₄ to about C₁₂;
c. an unsaturated alkyl chain having a chain length of from about C₄ to about C₁₂; and
d. combinations thereof
   to an area in need of treatment and, optionally, removing the composition and/or product following application. For clarity, the term “topically applying” is meant to refer to any of a number of techniques for applying the composition and/or product to a substrate, either animate or inanimate. For example, “topically applying” would include rubbing the compound on a surface, spraying it on a surface, applying the compound in a douche or in a lavage or any other technique that would bring the compound into contact with the microbes.

The method of inactivating viruses is useful in inactivating both enveloped and non-enveloped viruses. It is especially useful for quickly inactivating caliciform viruses such as norovirus. Furthermore, it is theorized that the utilization of this method will provide lasting residual efficacy on surfaces after they have been treated with the method of the present invention.

To reiterate, each of the methods of the present invention comprise the step of topically applying a composition or product comprising the disclosed composition to an area or surface in need of treatment. Examples of areas and/or surfaces in need of treatment, against which the compositions of the present invention are effective, include, but are not limited to: one or more hands, a nose, a nasal canal or passage, an article of clothing, a hard surface, a porous surface such as felt or wood, irritated, acne-affected, or injured skin, inflamed skin, pre or post surgical areas and combinations thereof.

The exact amount of antimicrobial composition and/or nature of a product will depend upon the needs and abilities of the formulator and practitioner of the present methods. Nevertheless, when the methods of the present invention are performed on a user's hands, for example, the antimicrobial compositions or products are applied in doses of from about 0.1 mL to about 5 mL per use, more preferably 0.5 mL to about 4 mL, most preferably from about 1 mL to about 3 mL. Once applied, the compositions may be rubbed on the treated surfaces for a period of time to ensure coverage, typically at least 5 seconds, preferably at least 10 seconds, more preferably at least 20 seconds and most preferably at least 30 seconds. If removal of the composition is desired, it is preferable to leave the composition on the surface for at least one minute, but it is unnecessary to leave the composition on the surface for more than 5 minutes or obtain effectiveness.

### PREPARATIVE EXAMPLES

In order to test the effectiveness of the methods of the present invention, two different sets of tests were performed. Three compositions were tested. Composition 1 is a control composition, while compositions 2 and 3 were prepared in accordance with the present disclosure.

Composition 1 was the control composition known as “ChloroPrep®).” ChloroPrep® is one of only two FDA NDA approved topical antiseptics. The ChloroPrep® composition was 70% isopropyl alcohol with 2% chlorhexidine gluconate (CHG) added to prevent bacterial growback. This compound is sold as a surgical site preparation alternative to iodophors, which are polymerized iodines.

Composition 2 was prepared in accordance with the present disclosure. Composition 2 comprised 1.5% C8AGS [CAS 51946-14-6], 8.5% sodium PCA (the sodium salt of pyroglutamic acid, specifically Anjidew NL50 [CAS 028874-51-3]) and 0.55% EHOP (ethylhexyloxypropanol, specifically Sensiva SC50 [CAS 70445-33-9]). Composition 2 was titrated to pH 3.0 with phosphoric acid.

Composition 3 was prepared in accordance with the present disclosure in accordance with the present invention. Composition 3 comprised 0.4% C8AGS [CAS 51946-14-6], 0.6% C8-10 methyl ester sulfonate (MES), 3.5% sodium PCA, 1.5% succinic acid [CAS 111015-6] and 0.5% EHOP [CAS 70445-33-9]. Composition 3 also included 1% potassium sorbate to increase antifungal activity and 0.35% parachlorometazylenol (PCMX), also known as chlorozylenol. Composition 3 was titrated to pH 3.0 with phosphoric acid.

Immediate Efficacy on Viruses

This was a standard suspension test for the efficacy of the methods of the present invention agents against a Hong Kong strain of influenza A virus, a WA strain of rotavirus, a HTLV-IIIB strain of HIV virus, feline calicivirus as a surrogate for norovirus, and avian influenza A virus done in accordance with ASTM E 1052. This suspension test measures the immediate effectiveness of the compounds on viruses. The log reduction in viral activity after one minute for each test is reported in Table 1.

TABLE 1

| | | |
|---|---|---|
| Product ID | Log Reduction 1 min. | Log Red. 5 min. |

Hong Kong strain of Influenza A virus (ATCC VR-544)

| | | |
|---|---|---|
| Composition 1 | 4.75 | 4.5 |
| Composition 2 | 3.75 | 3.5 |
| Composition 3 | 4.75 | 4.5 |

WA strain of Rotavirus (Ottawa, Ontario, Canada)

| | | |
|---|---|---|
| Composition 1 | 4.25 | 4.0 |
| Composition 2 | 5.25 | 5.0 |
| Composition 3 | 5.25 | 5.0 |

HTLV-IIIB strain of HIV virus (Advanced Biotechnologies)

| | | |
|---|---|---|
| Composition 1 | 3.0 | 3.0 |
| Composition 2 | 4.0 | 4.0 |
| Composition 3 | 4.0 | 4.0 |

Feline Calicivirus as a surrogate for norovirus
(F-9 strain ATCC VR-782)

| | | |
|---|---|---|
| Composition 1 | 4.25 | 5.25 |
| Composition 2 | 6.25 | 6.25 |
| Composition 3 | 6.25 | 6.25 |

Avian Influenza A virus (897/80-6750/78 strain ATCC VR-2072)

| | | |
|---|---|---|
| Composition 1 | 4.25 | 4.25 |
| Composition 2 | 5.25 | 5.25 |
| Composition 3 | 5.25 | 5.25 |

The results above show the method of the present invention has a surprisingly high immediate inactivating effect on feline calicivirus as well as other viruses.

Immediate and Residual Efficacy on Viruses

The second test was a novel protocol designed to test a practical clinical definition of hand hygiene persistence on a model of human skin. The FDA approved skin surrogate is called “VitroSkin®.” VitroSkin® is a complex, semiporous substance that includes collagen and lipids. VitroSkin® is much more difficult to disinfect than textiles or hard surfaces, because of the complex chemical interactions that can take place on its surface, as well as the presence of more places for microbes to avoid contact with compositions applied to the surface of the VitroSkin®.

In this experiment, for each virus tested, 1.0 to 1.5 inch square pieces of VitroSkin® were aseptically cut from a VitroSkin® sheet. The individual pieces were placed topography side up into individual petri dishes. A circle of approximately ½ inch diameter was drawn on the bottom of the VitroSkin® substrate to serve as a template for the test area. The test virus suspension was titered by 10-fold serial dilution and inoculated into the indicator cell cultures in quadruplicate on the day of test set-up to determine input virus titer.

A 0.025 mL aliquot of each test compound was applied to the surface of two skin substrate replicates, spread within the defined area, and allowed to dry for a specified time period of 1, 5, 60, 120, 240, 360, and 480 minutes at room temperature (23.0° C.). Following each drying period, the test virus suspension was thoroughly mixed and a 0.01 mL aliquot of the virus suspension was inoculated onto the surface of the VitroSkin® within the defined area. The virus remained in contact with the treated surface for a five minute exposure period at room temperature. Following the five minute exposure period, a sterile 1.5 mL cryovial containing 1.0 mL of elution medium was inverted over the defined area of each VitroSkin® substrate surface. The vial was held tightly against the surface, inverted and allowed to soak for a minimum of five seconds and inverted 20 times. The soak and inversion step was repeated one additional time. The vial was scraped gently across the surface to remove any excess test medium The solution was mixed using a vortex mixer and serial 10-fold dilutions were performed (0.1 mL+0.9 mL test medium). The dilutions were then assayed for presence of virus. The average percent reduction in viral activity and the average log₁₀ reduction for each virus is reported in Table 2 below.

TABLE 2
Influenza A

| | | | |
|---|---|---|---|
| Drying Parameter | Test virus | Average Percent | Average Log₁₀ |
| (minutes) | detected | Reduction | Reduction |

Test Substance: Compound 1

| | | | |
|---|---|---|---|
| 1 | YES | 24.1% | 0.1 |
| 5 | YES | 36.9% | 0.2 |
| 60 | YES | 71.8% | 0.55 |
| 120 | YES | 36.9% | 0.2 |
| 240 | YES | 49.9% | 0.3 |
| 360 | YES | 49.9% | 0.3 |
| 480 | YES | 68.4% | 0.5 |

Test Substance: Composition 2

| | | | |
|---|---|---|---|
| 1 | No | ≧99.9992% | ≧5.1 |
| 5 | No | ≧99.9992% | ≧5.1 |
| 60 | No | ≧99.9992% | ≧5.1 |
| 120 | No | ≧99.9992% | ≧5.1 |
| 240 | No | ≧99.9992% | ≧5.1 |
| 360 | No | ≧99.9992% | ≧5.1 |
| 480 | No | ≧99.9992% | ≧5.1 |

Test Substance: Compound 3

| | | | |
|---|---|---|---|
| 1 | No | ≧99.9995% | ≧5.3 |
| 5 | No | ≧99.9995% | ≧5.3 |
| 60 | No | ≧99.9995% | ≧5.3 |
| 120 | No | ≧99.9995% | ≧5.3 |
| 240 | No | ≧99.9995% | ≧5.3 |
| 360 | No | ≧99.9995% | ≧5.3 |
| 480 | No | ≧99.9995% | ≧5.3 |

Rotavirus

| | | | |
|---|---|---|---|
| Drying Parameter | Test virus | Average Percent | Average Log₁₀ |
| (minutes) | detected | Reduction | Reduction |

Test Substance: Compound 1

| | | | |
|---|---|---|---|
| 1 | Yes | 98.22% | 1.75 |
| 5 | Yes | 59.3% | 0.39 |
| 60 | Yes | 52.4% | 0.32 |
| 120 | Yes | 90.0% | 1.00 |
| 240 | Yes | 43.77% | 0.25 |
| 360 | Yes | 14.89% | 0.07 |
| 480 | Yes | 59.26% | 0.39 |

Test Substance: Compound 2

| | | | |
|---|---|---|---|
| 1 | No | 99.999% | 5.02 |
| 5 | No | 99.87% | 2.88 |
| 60 | No | 58.31% | 0.38 |
| 120 | No | 70.49% | 0.53 |
| 240 | No | No reduction | No reduction |
| 360 | No | 58.31% | 0.38 |
| 480 | No | 58.31% | 0.38 |

Test Substance: Compound 3

| | | | |
|---|---|---|---|
| 1 | Yes | ≧99.996% | 4.39 |
| 5 | Yes | 99.6% | 2.40 |
| 60 | Yes | 90.0% | 1.00 |
| 120 | Yes | 68.38% | 0.50 |
| 240 | Yes | 43.77% | 0.25 |
| 360 | Yes | 52.14% | 0.32 |
| 480 | Yes | 77.09% | 0.64 |

Avian Influenza A

| | | | |
|---|---|---|---|
| Drying Parameter | Test virus | Average Percent | Average Log₁₀ |
| (minutes) | detected | Reduction | Reduction |

Test Substance: Compound 1

| | | | |
|---|---|---|---|
| 1 | Yes | 98.38% | 1.79 |
| 5 | Yes | No reduction | No reduction |
| 60 | Yes | 68.4% | 0.5 |
| 120 | Yes | 62.85% | 0.43 |
| 240 | Yes | 75.45% | 0.61 |
| 360 | Yes | 86.20% | 0.86 |
| 480 | Yes | 68.4% | 0.50 |

Test Substance: Compound 2

| | | | |
|---|---|---|---|
| 1 | No | ≧99.9987% | ≧4.89 |
| 5 | No | ≧99.9987% | ≧4.89 |
| 60 | No | ≧99.9987% | ≧4.89 |
| 120 | No | ≧99.9987% | ≧4.89 |
| 240 | No | ≧99.9987% | ≧4.89 |
| 360 | No | ≧99.9987% | ≧4.89 |
| 480 | No | ≧99.9987% | ≧4.89 |

Test Substance: Compound 3

| | | | |
|---|---|---|---|
| 1 | No | ≧99.9987% | ≧4.89 |
| 5 | No | ≧99.9987% | ≧4.89 |
| 60 | No | ≧99.9987% | ≧4.89 |
| 120 | No | ≧99.9987% | ≧4.8 |
| 240 | Yes (one | 99.979% | 3.68 |
| | replicate) | | |
| 360 | No | ≧99.9987% | ≧4.89 |
| 480 | No | ≧99.9987% | ≧4.89 |

The results of this test also show that the method of the present invention exhibits surprisingly high log reductions in virus activity both immediately and up to eight hours after the method has been performed.

All documents cited are, in relevant part, incorporated herein by reference; the citation of any document is not to be construed as an admission that it is prior at with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of inactivating viruses, said method comprising the step of:
topically applying an antimicrobial composition comprising:
an organic acid; and
an anionic surfactant mixture having a characteristic selected from the group consisting of:
a. a linear alkyl chain having a chain length of from about C₄ to about C₁₂ and a total head group size of at least about 4 Angstroms;
b. a branched alkyl chain having a chain length of from about C₄ to about C₁₂;
c. an unsaturated alkyl chain having a chain length of from about C₄ to about C₁₂; and
d. combinations thereof.
to an area in need of treatment.

2. The method of claim 1, further comprising the steps of:
waiting at least one minute; and
removing said composition from said area.

3. The method of claim 2, wherein said antimicrobial composition retains antiviral efficiency on said surface for up to 8 hours.

4. The method of claim 1, wherein said viruses are enveloped viruses.

5. The method of claim 1, wherein said viruses are non-enveloped viruses.

6. The method of claim 1, wherein said virus to be inactivated at is a caliciform virus.

7. The method of claim 6, wherein said virus is norovirus.

8. A method of reducing the risk of viral infection and/or treating viral diseases in a mammal that may arise from said mammal's contact with a viral-infected surface, said method comprising the steps of:
topically applying an antimicrobial composition comprising:
an organic acid; and
an anionic surfactant mixture having a characteristic selected from the group consisting of:
a. a linear alkyl chain having a chain length of from about C₄ to about C₁₂ and a total head group size of at least about 4 Angstroms;
b. a branched alkyl chain having a chain length of from about C₄ to about C₁₂;
c. an unsaturated alkyl chain having a chain length of from about C₄ to about C₁₂; and
d. combinations thereof
to an area of said mammal that may or has come in contact with said surface; and
optionally, removing said composition following said application.

9. A method of reducing inflammation in a mammal, said method comprising the steps of:
topically applying an antimicrobial composition comprising:
an organic acid; and
an anionic surfactant mixture having a characteristic selected from the group consisting of:
a. a linear alkyl chain having a chain length of from about C₄ to about C₁₂ and a total head group size of at least about 4 Angstroms;
b. a branched alkyl chain having a chain length of from about C₄ to about C₁₂;
C. an unsaturated alkyl chain having a chain length of from about C₄ to about C₁₂; and
d. combinations thereof
to an inflamed area of said mammal in need of treatment.

10. The method of claim 9, further comprising the step of removing said composition following said application.

11. The method of claim 9, wherein said inflammation is caused by a source selected from the group consisting of plants, diaper rash, insect bites, allergic inflammatory reactions and combinations thereof.

12. (canceled)

13. (canceled)

14. (canceled)

15. A method of sanitizing mammalian skin, said method comprising the step of:
topically applying an antimicrobial composition comprising:
an organic acid; and
an anionic surfactant mixture having a characteristic selected from the group consisting of:
a. a linear alkyl chain having a chain length of from about C₄ to about C₁₂ and a total head group size of at least about 4 Angstroms;
b. a branched alkyl chain having a chain length of from about C₄ to about C₁₂;
c. an unsaturated alkyl chain having a chain length of from about C₄ to about C₁₂; and
d. combinations thereof
to an area of mammalian skin.

16. The method of claim 15, further comprising the step of removing said composition following said application.

17. The method of claim 16, wherein said mammalian skin is on human hands.

18. A method of manufacturing an antimicrobial wipe, said method comprising the steps of:
providing a substrate; and
saturating said substrate with an antimicrobial composition comprising:
an organic acid; and
an anionic surfactant mixture having a characteristic selected from the group consisting of:
a. a linear alkyl chain having a chain length of from about C₄ to about C₁₂ and a total head group size of at least about 4 Angstroms;
b. a branched alkyl chain having a chain length of from about C₄ to about C₁₂;
c. an unsaturated alkyl chain having a chain length of from about C₄ to about C₁₂; and
d. combinations thereof

19. The method of claim 18, wherein said substrate is paper.

20. The method of claim 19, wherein said substrate is cloth.

21. (canceled)

22. (canceled)

23. (canceled)

24. (canceled)

25. (canceled)

26. (canceled)

27. (canceled)

28. (canceled)

29. The method of claim 1 wherein said antimicrobial composition further comprises a nonionic agent.

30. The method of claim 29 wherein said nonionic agent is selected from the group consisting of 1-(2-ethylhexyl) glycerol ether, octyl glycerol ether, 2-(2-ethylhexylxoxy) propanol (EHOP), octyloxy propanol, 1-(2-ethylhexyloxy) ethanol, octyloxy ethanol, 1,2 hexylenediol, 1,2-cyclohexanedimethanol, isopropyl glycerol ether, 4-chloro-3-xylenol and combinations thereof.

31. The method of claim 1 wherein said virus to be inactivated is selected from the group consisting of Influenza A, Rotavirus, HIV virus, Feline Calicivirus, and Avian Influenza A virus.
